(19) United States
(12) Patent Application Publication
(10) Pub. No.: US 2018/0168770 A1
(43) Pub. Date: Jun. 21, 2018

(54) DEVICE AND METHOD FOR MARKING A LOCATION OF A TISSUE BIOPSY

(71) Applicant: RADVATION, LLC, Westmont, IL (US)

(72) Inventor: Allan M. Haggar, Westmont, IL (US)

(21) Appl. No.: 15/735,103

(22) PCT Filed: Jun. 9, 2016
(86) PCT No.: PCT/US16/36715

§ 371 Date: Dec. 8, 2017

Related U.S. Application Data
(60) Provisional application No. 62/267,745, filed on Dec. 15, 2015.
(60) Provisional application No. 62/242,186, filed on Oct. 15, 2015.
(60) Provisional application No. 62/174,367, filed on Jun. 11, 2015.

(51) Int. Cl. A61B 90/00

(52) U.S. Cl. CPC A61B 90/39; A61B 10/0283

(57) ABSTRACT

A device and method for marking the location of a tissue biopsy. The device includes a needle with a proximal end, a distal end, a slot disposed adjacent the distal end, and a tip having an opening. The device further includes a handle coupled to the proximal end of the needle, and a cutting apparatus. The cutting apparatus includes a cutting mechanism disposed within the needle and adapted to remove tissue from a target area. The device further includes an ejector mechanism having an ejector rod disposed within the needle and an actuator disposed on the handle and operatively coupled to the ejector rod. The actuator is actuated to eject a marker disposed within the distal end of the needle through one or more of the slot or the opening to mark the location of the tissue biopsy.

### CROSS-REFERENCE TO RELATED APPLICATIONS

This application claims the benefit of the filing date of U.S. Provisional Patent Application No. 62/174,367 filed Jun. 11, 2015; U.S. Provisional Patent Application No. 62/242,186 filed Oct. 15, 2015; and U.S. Provisional Patent Application No. 62/267,745 filed Dec. 15, 2015. Each of U.S. Provisional Patent Application No. 62/174,367, U.S. Provisional Patent Application No. 62/242,186, and U.S. Provisional Patent Application No. 62/267,745 is hereby incorporated by reference.

### TECHNICAL FIELD

This disclosure relates to biopsy methods and devices, and in particular, to an improved device and method for marking a location of a tissue biopsy.

### BACKGROUND

During ultrasound or mammography guided breast needle biopsies, a biopsy device is typically first inserted into and removed from a tumor and a separate device is then inserted into the same site to mark a location of an incision. Said another way, a first device is typically used to perform the biopsy and a second device, completely separate from the first device, is then inserted separately to deploy a marker into a target area of the tumor in which the biopsy was performed.

More specifically, and referring now to FIG. 1, in one example, a known biopsy device 10 is first inserted into a tumor target area 12 to perform a biopsy of one or more tissues in the tumor target area 12. The biopsy device 10 includes a needle 14 having a distal end 16, a proximal end 18 disposed opposite the distal end 16, and a slot 20 disposed at the distal end 16. A handle 22 is coupled to the proximal end 18 of the needle 14, and a tissue collection chamber 24 is removably coupled to a proximal end of the handle 22. The biopsy device 10 further includes a cutting apparatus 26 having a cutter drive 28 that extends from the distal end 16 of the needle 14 to the proximal end 18 of the needle 12. The cutting apparatus 26 further includes a cutter mechanism 30 disposed adjacent the distal end 16 of the needle 14 and coupled to the cutter drive 28. In operation, the cutting mechanism 30 rotates and advances in the slot 20 in the needle 14, and along with vacuum aspiration, withdraws tissue through the needle 14 to the tissue collection chamber 24 for later analysis.

Referring now to FIGS. 2A and 2B, after an adequate tissue sample has been withdrawn from the target 12, the biopsy device 10 is completely removed from the target area 12, and a marker deployment device 32 is inserted into the target area 12. The marker deployment device 32 is completely separate from the biopsy device 10 and used to deploy a marker, such as a marker clip, into the target area 12. More specifically, and in one example, the marker deployment device 32 is an end-loaded clip needle deployment device 32 having a needle 34, a marker clip 36 disposed at a distal end of the needle 34, and a plunger 38 disposed at a proximal end 40 of the device 32. Upon insertion of the needle deployment device 32 into the target area 12, the plunger 38 is in an extended position, as depicted in FIG. 2A, and the marker clip 36 is disposed within the needle 34. As depicted in FIG. 2B, when the plunger 38 is actuated, e.g., pushed toward the distal end of the needle deployment device 32, the marker clip 36 is ejected, e.g., pushed, out of a tip of the needle 34 to position the marker clip 36 in the target area 12.

In another example, and referring now to FIGS. 3A and 3B, an alternative marker deployment device 40 is depicted. More specifically, the marker deployment device 40 includes a catheter 41 having a preloaded marker clip 42 adjacent a distal end and a plunger 44 adjacent a proximal end. The marker deployment device 40 is adapted to be inserted into biopsy device 10 of FIG. 1, as depicted in FIG. 3B. More specifically, after the biopsy device 10 completes a biopsy of tissue in the target area 12 (FIG. 1), the biopsy device 10 is removed from the target area 12 and the cutting mechanism 26 is removed from the biopsy device 10. The marker deployment device 40 having the catheter 41 with the preloaded marker clip 42 is then inserted into the proximal end of the handle 22 of the biopsy device 10, as depicted in FIG. 3B. Initially, a head of the plunger 44 is in an extended position. The biopsy device 10 with the marker deployment device 40 inserted therein (and the cutting apparatus removed therefrom) may then be reinserted back into the target area 12 (FIG. 1) to deploy the marker 42 in the target area 12. More specifically, and as depicted in FIG. 3B, when the plunger 44 is actuated, such as pushed toward the distal end 16 of the needle 14 of the biopsy device 10, the marker clip 42 is forced or ejected out of the slot 20 and into the desired target area 12 of the tumor.

Both of the foregoing conventional methods of marking a target area of a tumor after a needle biopsy include a second device separate from the biopsy device. The second device is either itself inserted into the target area after the biopsy device is removed, as depicted in FIGS. 2A and 2B, or inserted into a biopsy device after the biopsy device is removed from the target area, as depicted in FIG. 3B. As a result, both methods require at least two steps. The first step includes insertion and removal of the biopsy device to complete the biopsy, and the second step includes reinsertion of either a completely separate marker device or a reconfigured biopsy device having a marker deployment device inserted therein. Said another way, in either of the foregoing described methods, the biopsy device must be first removed from the target area before any marker, such as a marker clip, may be deployed or inserted into the target area, e.g., an incision site. This additional step requires extra time for the procedure and risks potential error in insuring the marker deployment device is inserted into the same location or position of the incision, such that the marker is deployed in an accurate location. Further, potential errors may occur during any procedure between the first and second steps, such as problems with accurately removing the cutting apparatus from the biopsy device to accommodate the marker deployment device and incorrectly inserting the catheter marker deployment device into the biopsy device, for example. Such multiple opportunities for error and the lengthier process time of the conventional biopsy methods and devices increase the risks of improper markings in the target area and, ultimately, misdiagnosis.

### SUMMARY

A device and method for marking the location of a tissue biopsy is disclosed. In one example, the device comprises a needle including a proximal end, a distal end disposed opposite the proximal end, a slot disposed adjacent the distal end, and a tip disposed adjacent the distal end and having an opening. The biopsy device further comprises a handle coupled to the proximal end of the needle and a cutting apparatus including a cutter mechanism disposed adjacent the distal end of the needle and the slot, the cutting apparatus adapted to remove tissue from the target area. The device further includes ejector mechanism, the ejector mechanism including an ejector rod disposed within the needle and an actuator disposed on the handle and operatively coupled to the ejector rod. Upon removal of the tissue from the target area, the actuator of the ejector mechanism is actuated to eject a marker disposed within the distal end of the needle through one or more of the slot or the opening to mark a location of the tissue biopsy.

In another example, a device for marking the location of a tissue biopsy comprises a needle including a body with an upper chamber and a lower chamber. The needle further includes a proximal end, a distal end disposed opposite the proximal end, a slot disposed adjacent the distal end, and a tip disposed adjacent the distal end and having an opening. The device further includes a handle coupled to the proximal end of the needle, and a cutting apparatus including a cutter mechanism disposed within the upper chamber of the needle adjacent the distal end, the cutting apparatus adapted to remove tissue from the target area and the upper chamber adapted to receive tissue removed. The device also includes an ejector mechanism having an ejector rod disposed within the lower chamber of the needle and an actuator disposed on the handle and operatively coupled to the ejector rod. Upon removal of the tissue from the target area, the actuator of the ejector mechanism is actuated to eject a marker disposed within the lower chamber of the needle adjacent the distal end through the opening to mark a location of the biopsy.

In yet another example, a biopsy method comprises inserting a biopsy device into a target area, the biopsy device including a needle and a handle operatively coupled to a proximal end of the needle. The method further comprises actuating a cutting mechanism of a cutting apparatus disposed within the biopsy device to remove tissue from the target area, and actuating an actuator of one of an ejector mechanism or the cutting apparatus. The actuator is disposed on the handle, and the ejector mechanism includes an ejector rod disposed within the needle of the biopsy device and operatively coupled to the actuator. The method further comprises ejecting a marker disposed at the distal end of the needle through one or more of an opening of a tip of the needle or a slot disposed at the distal end of the needle to mark a point of incision in the target area.

In further accordance with any one or more of the foregoing examples or exemplary aspects, a device for marking a location of a tissue biopsy and/or a method for marking a location of a tissue biopsy optionally may include any one or more of the following further preferred forms.

In some preferred forms, the actuator may be disposed on one of a thumb side of the handle on or near a proximal end of the handle. In addition, the device for marking a location of a tissue biopsy may further include a cocking mechanism removably attached to the handle and having a chamber preloaded with a plurality of markers, the cocking mechanism coupled to the actuator, such that when the actuator is actuated, one marker from the plurality of markers disposed within the cocking mechanism is adapted to be removed from the cocking mechanism, pushed into the needle, and ejected out of one of the slot or the opening disposed at the distal end of the needle. The cocking mechanism may be coupled to the handle at one of a proximal end of the handle or between the proximal end of the needle and the proximal end of the handle.

In some preferred forms, the marker may be one or more of a marker clip, a marker label, or a marker tag. The marker may include dissolvable material not requiring removal from the target area. The cutting apparatus may include a cutter drive disposed within a body of the needle, and the cutter drive may be disposed adjacent to the ejector rod. In addition, the handle may further comprise a motor disposed near the proximal end of the handle, and the motor may be operatively coupled to the cutting apparatus to actuate the cutting mechanism during the biopsy.

In some preferred forms, the device may further comprise an undersurface slot on an undersurface of the needle, the undersurface slot opening into the needle, wherein when the actuator is actuated, the marker is ejected through the undersurface slot to mark the location of the tissue biopsy. In addition, the tip of the cutting-apparatus may be spring-loaded.

In some other preferred forms, the actuator may be a winding mechanism having a depressor disposed in a center of the winding mechanism, and a spring may be disposed on the ejector rod near the proximal end of the handle. The spring may be operatively coupled to the actuator such that when the winding mechanism is rotated in a clockwise direction the actuator is in a spring-loaded position, and when the depressor is pushed the spring and the ejector rod move in a direction toward the distal end of the needle to eject the marker through one of the slot or the opening.

In some preferred forms, the device may further comprise a racking cocking mechanism. The racking cocking mechanism may have a side slot disposed on a side portion of the handle, and a finger trigger disposed within the side slot and operatively coupled to the ejector rod. Movement of the finger trigger in a direction toward the proximal end of the handle puts the ejector rod and finger trigger in a cocked position. Pushing the actuator button in a direction toward the distal end of the needle moves the ejector rod and finger trigger to an uncocked position to eject the marker through one or more of the slot or the opening.

In some other preferred forms, the method may further comprise preloading at least one marker adjacent the distal end of the needle before inserting the biopsy device into the target area. In addition, preloading the at least one marker may comprise inserting a catheter with the at least one marker into the needle until the at least one marker is positioned adjacent the distal end of the needle. Further, actuating the actuator of one of the ejector mechanism or the cutting apparatus may comprise removing at least one marker from a plurality of markers preloaded in a chamber of a cocking mechanism coupled to the handle, and pushing the at least one marker adjacent the distal end of the needle. Still further, actuating the actuator of the ejector mechanism may comprise pushing the actuator, thereby moving the ejector rod in a longitudinal direction toward the distal end of the needle to contact the marker. Ejecting a marker disposed adjacent the distal end of the needle may comprise ejecting one of a marker clip, a marker tag, a marker post, a marker label and/or any other marker adapted to mark a location of a tissue biopsy.

Additional optional aspects and features are disclosed, which may be arranged in any functionally appropriate manner, either alone or in any functionally viable combination, consistent with the teachings of the disclosure. Other aspects and advantages will become apparent upon consideration of the following detail description.

### BRIEF DESCRIPTION OF THE DRAWINGS

FIG. 1 is a perspective view of a conventional biopsy device;

FIG. 2A is a perspective view of a conventional marker clip deployment device in which a marker clip disposed within the marker clip deployment device is not ejected into a target area;

FIG. 2B is a perspective view of a conventional marker clip deployment device in which a marker clip disposed within the marker clip deployment device is ejected into a target area;

FIG. 3A is a perspective view of a conventional catheter having a marker clip, the catheter adapted to be inserted into the conventional biopsy device of FIG. 1;

FIG. 3B is a perspective view of the conventional catheter of FIG. 3A inserted into a conventional biopsy device;

FIG. 4 is a perspective view of a biopsy device according to one aspect of the present disclosure;

FIG. 5A is a close-up view of a distal end of a needle of the biopsy device of FIG. 4, the biopsy device having a preloaded marker clip adapted to be ejected out of an opening in a needle of the biopsy device;

FIG. 5B is a cross-sectional view of a portion of the needle of FIG. 5A taken along the line A-A in FIG. 5A;

FIG. 6 is another close-up view of a distal end of a needle of the biopsy device of FIG. 4, the biopsy device having a preloaded marker clip adapted to be ejected out of a slot in the needle of the biopsy device;

FIG. 7A is a perspective view of a biopsy device according to another aspect of the present disclosure;

FIG. 7B is a perspective view of a biopsy device according to another aspect of the present disclosure;

FIG. 8A is a perspective view of a biopsy device according to another aspect of the present disclosure;

FIG. 8B is a cross-sectional view of a portion of the needle of FIG. 8A taken along the line D-D in FIG. 8A;

FIG. 9 is a perspective view of a biopsy device according to another aspect of the present disclosure;

FIG. 10 is a perspective view of a biopsy device according to another aspect of the present disclosure

FIG. 11A is a perspective view of a biopsy device according to yet another aspect of the present disclosure;

FIG. 11B is a rear view of the biopsy device of FIG. 11A;

FIG. 12A is a perspective view of a biopsy device according to yet another aspect of the present disclosure, the biopsy device including a cocking mechanism in an unextended position; and

FIG. 12B is another perspective view of the biopsy device of FIG. 12A, the cocking mechanism in an extended position.

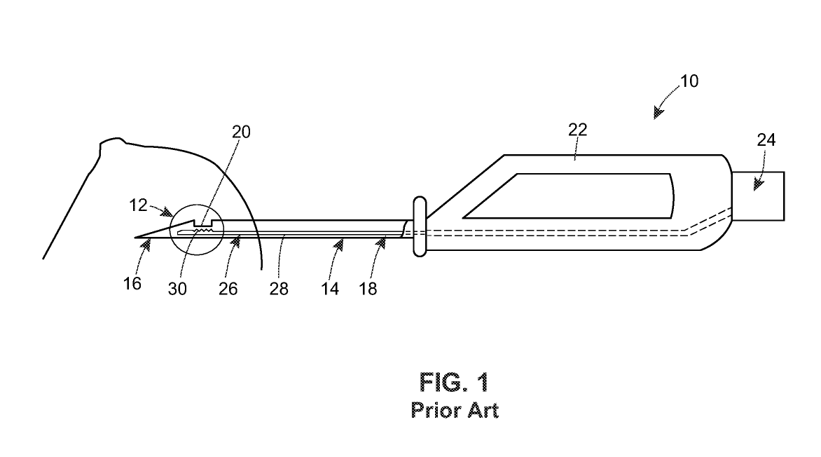

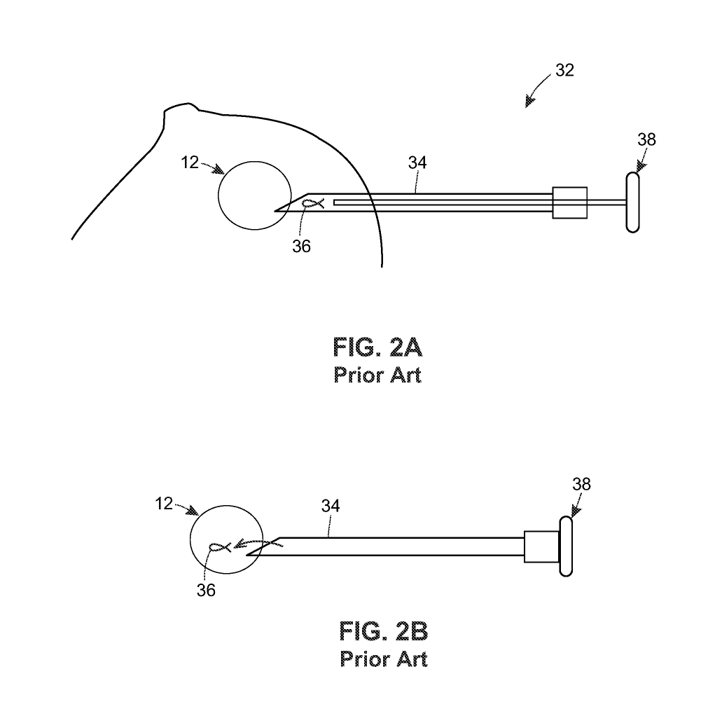

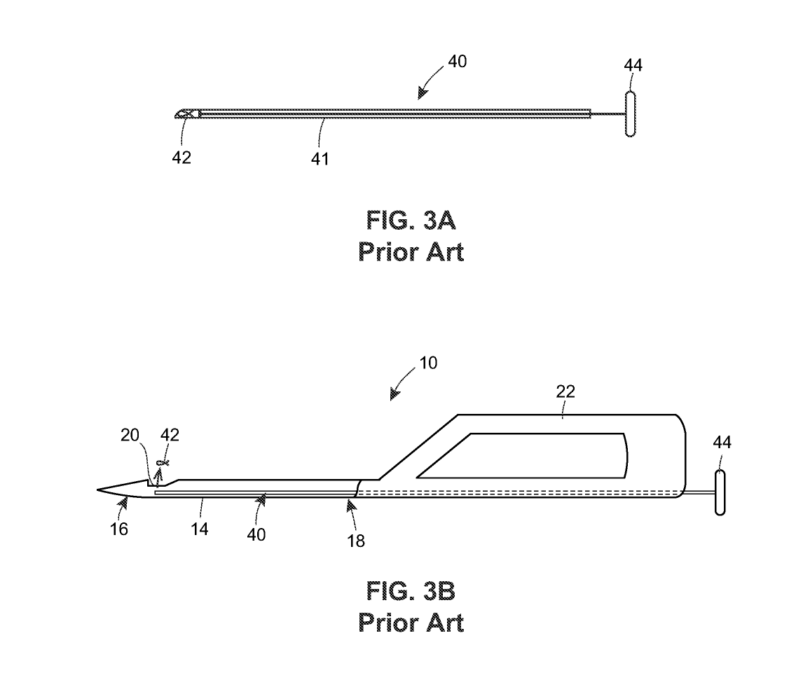

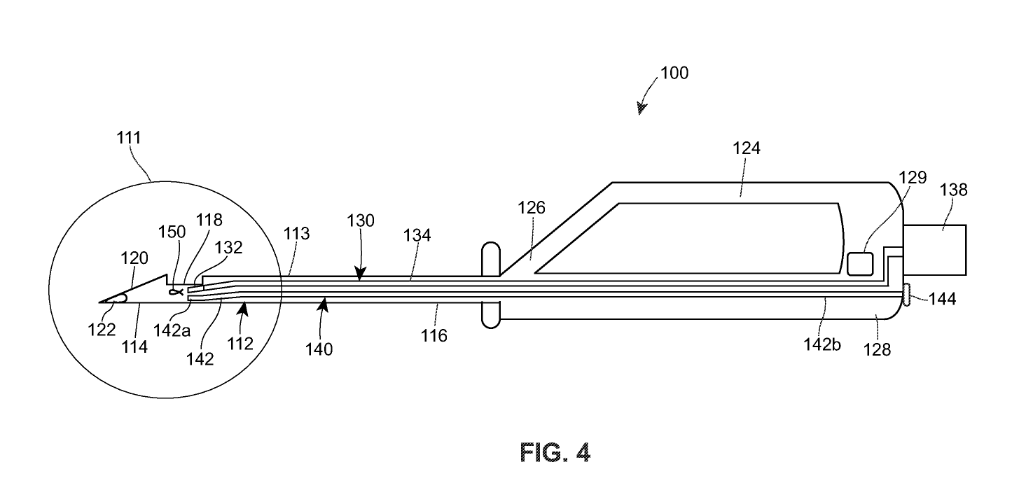

### DETAILED DESCRIPTION

Generally, a device and method for marking the location of a tissue biopsy are disclosed. The device comprises a needle preloaded with a marker, such as a marker clip, adapted to mark a point of incision in one or more tissues of a target area. The device further includes a handle coupled to the needle at a proximal end of the needle and a cutting apparatus adapted to remove tissue from the target area. The cutting apparatus includes a cutter mechanism disposed adjacent a distal end of the needle and the slot. The device also includes an ejector mechanism having an ejector rod disposed within the needle and an actuator disposed on the handle and operatively coupled to the ejector rod. Upon completion of the biopsy, the biopsy device is not removed from a target area of the tumor, but instead remains inserted in the target area. Rather, the actuator is actuated to eject the marker clip through one or more of a slot or an opening disposed at a distal end of the needle to mark the point of incision in the target area. Thus, the device of the present disclosure allows for a one-step insertion process in the target area. Said another way, the new device is only inserted into the target area one time to both perform the biopsy and deploy the marker in the target area without having to remove the biopsy device from the target area at any time. In other words, the device and method of the present disclosure deploy a marker into the target area through a device without having to remove the device from the target area after completion of the biopsy and then reintroduce a second marker deployment device, as in conventional biopsy methods and devices.

Referring now to FIG. 4, a biopsy device 100 of the present disclosure adapted to be inserted into a target area 111 of a tumor, for example, is depicted. The biopsy device 100 includes a needle 112 having a body 113 with a distal end 114 and a proximal end 116 disposed opposite the distal end 114. A slot 118, such as a cutter slot, is disposed adjacent the distal end 114 of the needle 112 and on an outer side of the needle 112. While the slot 118 is generally rectangular in shape, one of ordinary skill in the art will appreciate that the slot 118 may alternatively be various other shapes, such as one or more of semi-circular, circular, or triangular in shape, for example, and still fall within the scope of the present disclosure. The needle 112 further includes a tip 120 also disposed adjacent the distal end 114 of the needle 112. The tip 120 includes an opening 122 adapted to receive a marker, such as a marker clip 150, in one example, as described in more detail below.

The biopsy device 100 further includes a handle 124 having a distal end 126 that is coupled to the proximal end 116 of the needle 112, and a proximal end 128. A biopsy motor 129 may be disposed within the handle 124 at the proximal end 128 to provide power to a vacuum (not shown), for example, and/or a cutting apparatus of the biopsy device 100, as described more below. The vacuum may provide a suction force at the proximal end of the needle 112 to withdraw tissue from the point of incision in the target area 111 into the needle 112. While the handle 124 is depicted as generally rectangular in shape in FIG. 4, the handle 124 may take the shape of one or more of a variety of shapes, such as a semi-circle, a circle, an oval, or a triangle, and still fall within the scope of the present disclosure.

As further depicted in FIG. 4, the biopsy device 100 also includes a cutting apparatus 130 disposed within the body 113 of the needle 112. The cutting apparatus 130 includes a cutting mechanism 132 disposed adjacent the distal end 114 of the needle 112 and a cutter drive 134 coupled to the cutting mechanism 132. In one example, the cutting mechanism 132 is disposed adjacent to the slot 118, such that the cutting mechanism 132 rotates and advances in the slot 118 during operation of the biopsy device 100 to remove tissue from the target area 111 through the slot 118, for example. The cutter drive 134 extends along the length of the needle 112 of the biopsy device 100 from the cutting mechanism 132 disposed at the distal end 114 of the needle 112 to the proximal end 116 of the needle 112. In addition, a tissue recovery chamber 138 is disposed at the proximal end 128 of the handle 124 for receiving excised tissue from the needle 112.

The biopsy device 100 further includes an ejector mechanism 140 having an ejector rod 142 disposed within the body 113 of the needle 112 and an actuator 144, such as an actuator button, disposed on the handle 124 and operatively coupled to the ejector rod 142. In one example, and as depicted in FIG. 4, the actuator button 144 is disposed on a rear end of the handle 124 near the proximal end 128 of the handle 124 for ease of actuation during use of the biopsy device 100. Alternatively, the actuator button 144 may be disposed on another side or area of the handle 124 and still fall within the scope of the present disclosure.

In one example, the actuator 144 is a plunger-type mechanism. In this example, the actuator 144 plunger is initially in an extended position, such as at the beginning of and through the biopsy procedure. When it is time to deploy the pre-loaded marker 150 disposed in the needle 112 through one or more of the slot 118 or the opening 122, the plunger of the actuator 144 is pushed in a direction toward the distal end 114 of the needle 112. A distal end of the plunger of the actuator 144 then contacts the marker 150 upon actuation, e.g., being pushed, to move the marker 150 one of through the slot 118 or the opening 122 and mark the location of the biopsy.

In another example, the actuator 144 is spring-loaded. In this example, the ejector rod 142 has a distal end 142a and a proximal end 142b. The proximal end 142b or the actuator button 144 may include a spring biased in a direction toward the proximal end 128 of the handle 124. When it is time to deploy the pre-loaded marker 150, the actuator 144 button is pushed, which counteracts and overcomes the force of the loaded spring or springs (not shown) disposed at the proximal end 142 of the ejector rod 142 or within the actuator button 144. The one or more springs then extend in a direction toward the distal end 114 of the needle 112 to move the ejector rod 142 in a direction toward the distal end 114 of the needle 112. The ejector rod 142 is moved until contacting the marker 150, which is then ejected out of the distal end 114 of the needle 112 through one or more of the opening 122 or the slot 118 to mark a location of the tissue biopsy.

While the foregoing example actuators 144 may be used to eject the marker 150 out of the needle 112, for example, one of ordinary skill in the art will appreciate that various other actuating mechanisms may alternatively and/or additionally be used as other example actuators 144 and still fall within the scope of the present disclosure.

The needle 112 of the biopsy device 100 may include one of a 10-gauge, an 11-gauge, a 12-gauge, a 13-gauge, or a 14-gauge aperture disposed along the length of the body 113, such that an adequate amount of tissue extracted from the target area 111 by the cutting mechanism 132 may be suctioned through the needle 112. In addition, the aperture of the body 113 of the needle 112 must further include a diameter large enough to accommodate both the ejector rod 142 of the ejector mechanism 140 and the cutting mechanism 130, as depicted in FIG. 4, for example.

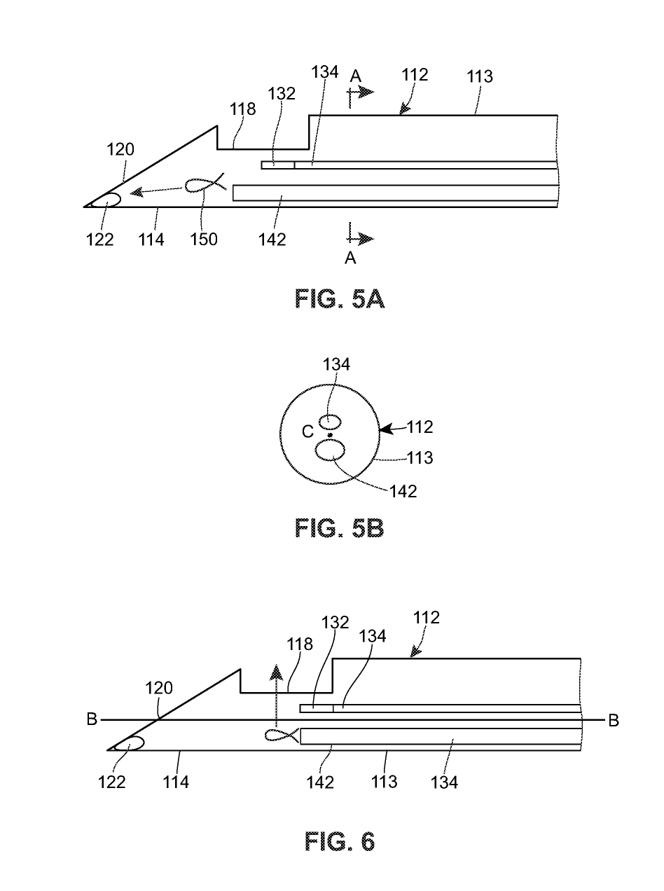

Referring now to FIGS. 5A and 5B, upon completion of a biopsy, e.g., when an adequate amount of tissue from the target area 111 of the tumor has been extracted through the needle 112, the actuator 144 (FIG. 4) may be actuated to mark a location of a tissue biopsy, e.g., to position a marker at the point of incision at the target area 111 of the tumor. More specifically, a marker, such as the marker clip 150, is preloaded in the distal end 114 of the needle 112. Upon actuation of the actuator 144 (FIG. 4), the actuator rod 142 moves toward the distal end 114 of the needle 112 to contact the marker clip 150 and push and/or eject the marker clip 150 through one of the slot 118 or the opening 122. As depicted in FIG. 5A, the ejector rod 142 pushes the marker clip 150 in a longitudinal direction toward the opening 122 in the tip 120 of the needle 112. The ejector rod 142 will continue to do so until the marker clip 150 is ejected through the opening 122 and into the target area 111 of the tumor.

FIG. 5B is a cross-section of the needle 112 taken along the line A-A in FIG. 5A and depicts a cross-sectional view of the ejector rod 142 and the cutter drive 134 disposed within a bore of the needle 112. In one example, and as depicted, the ejector rod 142 is disposed adjacent to and below the cutter driver 134 on either side of a center C of the bore of the needle 112. In addition, the diameter of the needle 112 is large enough to accommodate both the ejector rod 142 and the cutter drive 134, such that the ejector rod 142 does not contact any surface of an inside surface of the body 113 of the needle 112 or any surface of the cutter drive 134. In a similar manner, the cutter drive 134 does not contact the body 113 of the needle 112 or the ejector rod 142. Such a configuration helps enable a smooth and resistance free biopsy and marking process for the device of the present disclosure, for example.

As depicted in FIG. 6, the ejector rod 142 may alternatively push the marker clip 150 in a direction perpendicular to a longitudinal axis B-B of the needle 112, until the marker clip 150 is ejected through the slot 118 and into a desired point in the target area 111 of the tumor. In one example, the actuator 144 may include a plunger that is first pushed in a longitudinal direction toward the distal end 114 of the needle 112 until contacting the marker 150. Upon contact with the marker 150, for example, the plunger of the actuator 144 may be moved in an upward direction, such that the distal end 142a of the ejector rod 142 is disposed at least partially or completely below the marker 150. The plunger of the actuator 144 may then be moved in a downward direction to push the marker 150 in a direction toward the slot 118, such as perpendicular to the longitudinal axis B-B of the needle 112, and ultimately through the slot 118. The marker clip 150 may also be pushed in any other direction that is less than perpendicular to the longitudinal axis B-B of the needle 112 and not parallel to the longitudinal axis B-B that allows that marker clip 150 be adequately pushed through the slot 118.

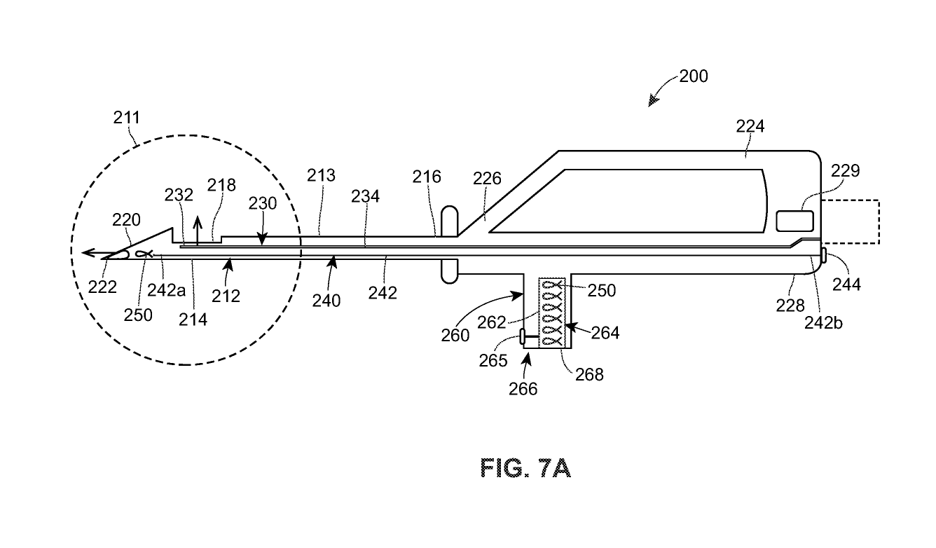

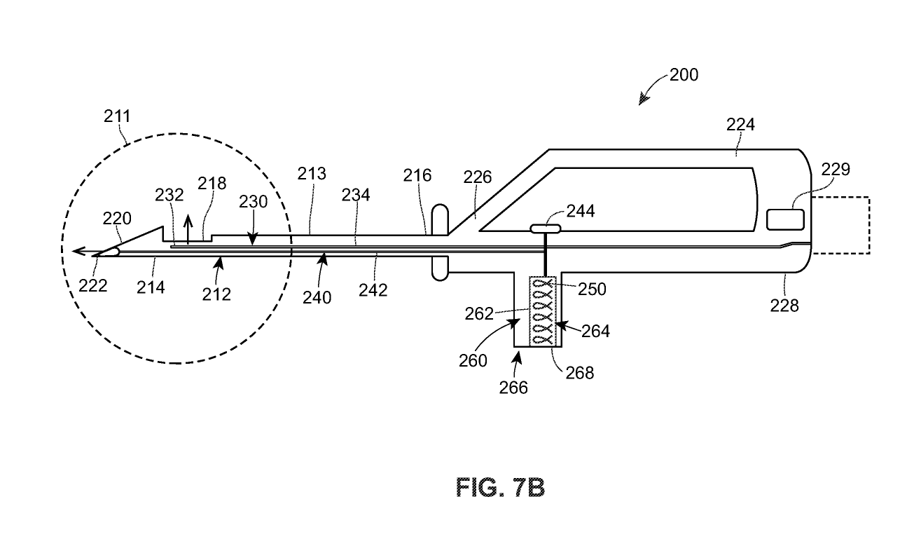

Referring now to FIG. 7A, a biopsy device 200 according to another aspect of the present disclosure is depicted. The biopsy device 200 is similar to the biopsy device 100 of FIGS. 4-6, but further includes a cocking mechanism 260 for storing markers, such as marker clips, to be preloaded into the needle, as described more below. Elements and structural components of the biopsy device 200 common or similar to elements and structural components of the biopsy device 100 include reference numerals 100 more than the biopsy device 100.

For example, like the biopsy device 100 of FIG. 4, the biopsy device 200 is adapted to be inserted into a target area 211 of a tumor, for example. The biopsy device 200 also includes a needle 212 having a body 213 with a distal end 214 and a proximal end 216 disposed opposite the distal end 214. A slot 218 is disposed adjacent the distal end of the needle 212, and the needle 212 further includes a tip 220 having an opening 222. While the slot 218 of the biopsy device 200 is disposed on an outer top surface of the body 213 of the needle 212 and is generally rectangular in shape, one of ordinary skill in the art will appreciate that the slot 218 may alternatively be disposed on an outer bottom surface of the body 213 and/or be semi-circular, circular, triangular, or rounded in shape and still fall within the scope of the present disclosure.

As further depicted in FIG. 7A, the biopsy device 200 also includes a handle 224 having a distal end 226 coupled to the proximal end 216 of the needle 212 and a proximal end 228 Like the handle 112 of the biopsy device 100, the handle 212 may also include a motor 229 adapted to drive a cutting mechanism and a vacuum (not shown) disposed within the biopsy device 200. In addition, the biopsy device also includes an ejector mechanism 240 having an ejector rod 242 disposed within the body 213 of the needle 212 and an actuator 244, such as an ejector button, disposed on the handle 224 and operatively coupled to the ejector rod 242.

In addition, the actuator 244 may include a plunger-type mechanism. In this example, the actuator 244 plunger is initially in an extended position, such as at the beginning of and through the biopsy procedure. When it is time to deploy a pre-loaded marker 250 disposed in the needle 212 through one or more of the slot 218 or the opening 222, the plunger of the actuator 244 is pushed in a direction toward the distal end 214 of the needle 212. A distal end of the ejector rod 242 then contacts the marker 250 upon actuation to move the marker 250 one of through the slot 118 or the opening 122 and mark the location of the biopsy.

In yet another example, the actuator 244 may be spring-loaded. In this example, the ejector rod 242 has a distal end 242a and a proximal end 242b. The proximal end 242b or the actuator button 244 may include a spring (not shown) biased in a direction toward the proximal end 228 of the handle 224. When it is time to deploy the pre-loaded marker 250, the actuator 244 button is pushed, which counteracts and overcomes the force of the loaded spring or springs (not shown) disposed at the proximal end 242b of the ejector rod 242 or within the actuator button 244. The one or more springs then extend in a direction toward the distal end 214 of the needle 212 to move the ejector rod 242 in a direction toward the distal end 214 of the needle 212. The ejector rod 242 is moved until contacting the marker 250, which is then ejected out of the distal end 214 of the needle 212 through one or more of the opening 222 or the slot 218 to mark a location of the tissue biopsy.

While the foregoing example actuator 244 may be used to eject the marker 250 out of the needle 212, for example, one of ordinary skill in the art will appreciate that various other actuating mechanisms, such as any of the actuating mechanisms disclosed in other examples of the application, may alternatively and/or additionally be used and still fall within the scope of the present disclosure. For example, and as depicted in FIG. 7B, the actuator 244 may alternatively be disposed near the distal end 226 of the handle 224 on an inside surface of the handle 224. This example actuator 244 is operatively coupled to the ejector rod 242, e.g., may be spring-loaded, such that upon actuation of the actuator 244, the ejector rod 244 is moved until contacting the preloaded marker 250 to eject the marker 250 out of the needle 212.

A cocking mechanism 260 is removably attached to the handle 224 and has a chamber 262 preloaded with a plurality of markers 264, such as marker clips 250. The chamber 262 may be cylindrical or rectangular in shape or any combination of one or more other shapes such that the plurality of markers 264 disposed therein is able to be in a stacked configuration for easy insertion and removal. In addition, while the cocking mechanism 260 is depicted as rectangular in shape, one of ordinary skill in the art will understand that the cocking mechanism 260 may alternatively take the form of one or a combination of other shapes, such as a circle, a semi-circle, a square or a triangle, and still fall within the scope of the present disclosure.

The cocking mechanism 260 includes an end 266 having a cover 268 for enclosing the chamber 262 and the marker clips 250 disposed therein. The cover 268 helps prevent the marker clips 250 from inadvertently falling out of the end 266 of the cocking mechanism 260 during operation of the biopsy device 200. In addition, the cover 268 may be easily removed from the end 266 of the chamber 262 to allow additional marker clips 250 to be loaded in a stacked configuration into the chamber 262 of the cocking mechanism 260 after ejection of all or most markers 264 of the cocking mechanism 260. In one example, the cover 268 may be attached to the end of the cocking mechanism 260 in a hinged configuration. In another example, the cover 268 may be attached to the end of the cocking mechanism 260 in a sliding configuration, the sliding configuration one in which the cover 268 is slidably removable from the end 266 of the chamber 262.

The cocking mechanism 260 may be coupled to the handle 224 below the actuator button 244 and between the proximal end 216 of the needle 212 and the proximal end 228 of the handle 224, as depicted in FIGS. 7A and 7B. Alternatively, the cocking mechanism 260 may be coupled to the handle 224 at the proximal end 228 of the handle 224.

In one example, the cocking mechanism 260 includes an actuator 265 disposed on an outside surface, as depicted in FIG. 7A, for example. The actuator 265 may be spring-loaded and is coupled to the plurality of markers 264. Upon actuation of the actuator 265, the actuator 265 moves in a direction toward the plurality of markers 264 and at least one marker 250 of the plurality of markers 264 is pushed upward and into the needle 212. Then, upon a first actuation of the actuator button 244 that is operatively coupled to the ejector rod 242, the at least one marker 250 of the plurality of markers 264 is moved in a longitudinal direction toward the distal end 214 of the needle 212. Upon a second actuation of the actuator 244, the marker 250 is ejected out of one of the slot 218 or the opening 222 disposed at the distal end 214 of the needle 212 to mark the location of the biopsy. In another example, the at least one marker clip 250 is ejected into the needle 212 and pushed to the distal end 214 of the needle 212 upon a first actuation of the actuator button 244. Upon a second actuation of the actuator button 244, the marker clip 250 is then ejected out of one of the slot 218 or the opening 222 disposed at the distal end 214 of the needle 212.

While the actuator 265 disposed on the outside surface of the cocking mechanism may be spring loaded, e.g., include at least one spring biased in the direction of the distal end 214 of the needle 212, one of ordinary skill in the art will appreciate that the actuator 265 may alternatively and/or additionally include various other actuating mechanisms and still fall within the scope of the present disclosure.

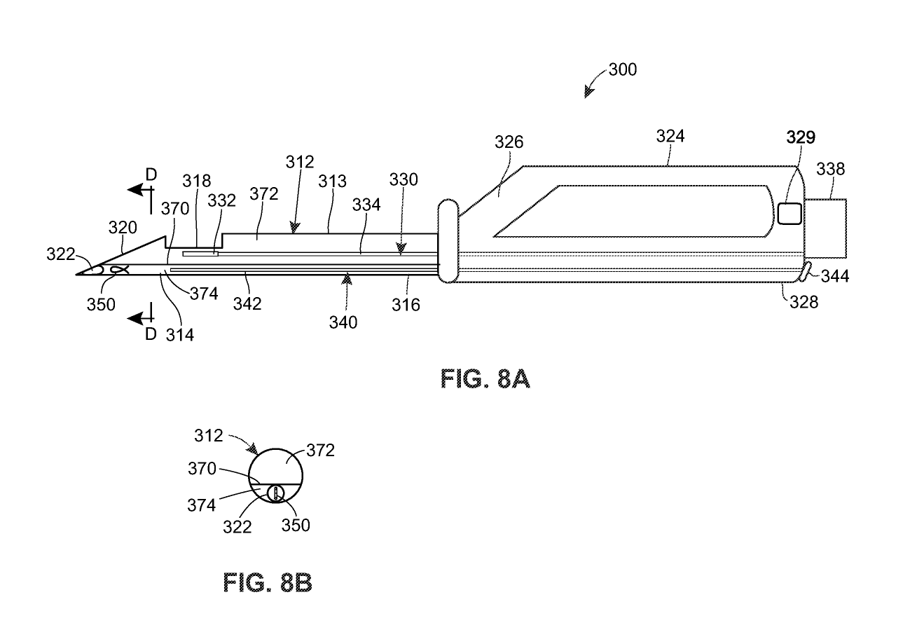

Referring now to FIG. 8A, a biopsy device 300 according to another aspect of the present disclosure is depicted. The biopsy device 300 is similar to the biopsy devices 100, 200, but also includes a double barrel needle, as explained more below. Elements and structural components of the biopsy device 300 common or similar to elements and structural components of the biopsy device 100, 200 include reference numerals 200 more than the biopsy device 100 and 100 more than biopsy device 200.

For example, like the biopsy device 100 of FIG. 4, the biopsy device 300 is adapted to be inserted into a target area (not shown) of a tumor, for example. The biopsy device 300 also includes a needle 312 having a body 313 with a distal end 314 and a proximal end 316 disposed opposite the distal end 314. A slot 318 is disposed adjacent the distal end 314 of the needle 312, and the needle 312 further includes a tip 320 having an opening 322. While the slot 318 of the biopsy device 300 is disposed on an outer surface of the body 313 of the needle 312 and is generally rectangular in shape, one of ordinary skill in the art will appreciate that the slot 318 may alternatively be semi-circular, circular, triangular, or rounded in shape and still fall within the scope of the present disclosure.

As further depicted in FIG. 8A, the biopsy device 300 also includes a handle 324 having a distal end 326 coupled to the proximal end 316 of the needle 312 and a proximal end 328 Like the handle 112 of the biopsy device 100, the handle 312 may also include a motor 329 (not shown) adapted to drive a cutting mechanism and a vacuum (not shown) disposed within the biopsy device 200, such as the handle 224.

In addition, and unlike the needles 112, 212 of the previous example devices, the needle 312 of the device 300 has a double barrel configuration. More specifically, and as depicted in both FIGS. 8A and 8B, the needle 312 includes a septum 370 disposed within a bore of the body 313 of the needle 312. The septum 370 extends along the length of the needle 312 from the distal end 314 to the proximal end 316, creating an upper chamber 372 and a lower chamber 374 within the needle 312. The upper chamber 372 receives a cutting mechanism 330 and tissue excised from a target area during the biopsy. The lower chamber 374 is disposed below and opposite the upper chamber 372 and includes a preloaded marker 350 and an ejector rod 342 of an ejector mechanism 340. The ejector mechanism 340 includes an actuator 344, such as an actuator button, disposed on the handle 324 and operatively connected to the ejector rod 342. The actuator 344 may include one or more of a plunger mechanism or a spring-load actuator 344, such as the actuators 144, 244 previously described, and, therefore, further operates like the actuators 144, 244 described above. In this example, the upper chamber 372 is larger than the lower chamber 374, such that the upper chamber 372 is able to receive a significant amount of excised tissue during a biopsy. In one example, the upper chamber 372 includes one of a 10-gauge, an 11-gauge, a 12-gauge, a 13-gauge, or a 14-gauge aperture, and the lower chamber 374 includes any of one of a 14-gauge aperture through a 22-gauge aperture, or a similar size just large enough to accommodate the ejector rod 342. Said another way, the lower chamber 374 may include any one of a 14-gauge, a 15-gauge, a 16-gauge, a 17-gauge, an 18-gauge, a 19-gauge, a 20-gauge, a 21-gauge or a 22-gauge aperture. As one of ordinary skill in the art will appreciate, the upper and lower chambers 372, 374 may alternatively include various other sizes of apertures, in particular variations in which the upper chamber 372 is larger than the lower chamber 374, and still fall within the scope of the disclosure.

Upon completion of a biopsy, e.g., when an adequate amount of tissue from the target area of the tumor has been extracted through the upper chamber 372 needle 312, the actuator 444 may be actuated to position a marker at the point of incision at the target area of the tumor. More specifically, a marker, such as the marker clip 350, is preloaded in the distal end 314 of the needle 312. Upon actuation of the actuator 344 (FIG. 8A), the ejector rod 342 moves toward the distal end 314 of the needle 312 to contact the marker clip 350 and push and/or eject the marker clip 350 through the opening 322 and into the target area of the tumor.

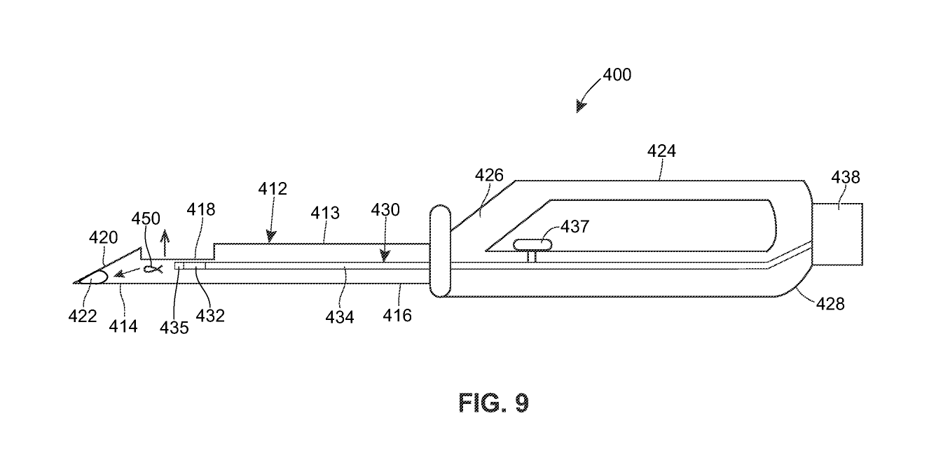

Referring now to FIG. 9, a biopsy device 400 according to another aspect of the present disclosure is depicted. The biopsy device 400 is similar to the biopsy devices 100, 200, 300, but also includes a tip of a cutting apparatus that is actuated to push a pre-loaded marker out of a needle, as explained more below. Elements and structural components of the biopsy device 400 common or similar to elements and structural components of the biopsy device 100, 200, 300 include reference numerals 300 more than the biopsy device 100; 200 more than biopsy device 200; and 100 more than biopsy device 300.

For example, like the biopsy device 100 of FIG. 4, the biopsy device 400 is adapted to be inserted into a target area (not shown) of a tumor, for example. The biopsy device 400 also includes a needle 412 having a body 413 with a distal end 414 and a proximal end 416 disposed opposite the distal end 414. A slot 418 is disposed adjacent the distal end 414 of the needle 412, and the needle 412 further includes a tip 420 having an opening 422. While the slot 418 of the biopsy device 400 is disposed on an outer top surface of the body 413 of the needle 412 and is generally rectangular in shape, one of ordinary skill in the art will appreciate that the slot 318 may alternatively be semi-circular, circular, triangular, or rounded in shape and still fall within the scope of the present disclosure.

As further depicted in FIG. 9, the biopsy device 400 also includes a handle 424 having a distal end 426 coupled to the proximal end 416 of the needle 412 and a proximal end 428 Like the handle 124 of the biopsy device 100, the handle 424 may also include a motor (not shown) adapted to drive a cutting mechanism and a vacuum (not shown) disposed within the biopsy device 400.

The biopsy device 400 also includes a cutting apparatus 430. The cutting apparatus 430 includes a cutting mechanism 432 disposed adjacent the distal end 414 of the needle 412 and a cutter drive 434 coupled to the cutting mechanism 432. In one example, the cutting mechanism 432 is disposed adjacent to the slot 418, such that the cutting mechanism 432 rotates and advances in the slot 418 during operation of the biopsy device 400 to remove tissue from the target area through the slot 418, for example. The cutter drive 434 extends along the length of the needle 412 of the biopsy device 400 from the cutting mechanism 432 disposed at the distal end 414 of the needle 412 to the proximal end 416 of the needle 412. In addition, a tissue recovery chamber 438 is disposed at the proximal end 428 of the handle 424 for receiving excised tissue from the needle 412.

In the biopsy device 400, however, an ejector mechanism is not disposed within the needle 412 and handle 424, for example. Instead, the cutting apparatus 430 includes a tip 435 disposed adjacent to and connected to the cutting mechanism 432. The tip 435 includes a spring that is biased toward the proximal end 416 of the needle 412. An actuator 437 is disposed on the handle 424 of the device 400 and operatively coupled to the cutting mechanism 432. The tip 437 is actuated by the actuator 437 after a desired amount of tissue is excised from the target area to move the tip 437 into contact with a marker 450 preloaded in the needle 430 adjacent the distal end 414. More specifically, upon actuation of an actuator 437, such as an actuator button, disposed, for example, on an inside surface of the handle 424, the tip 435 moves toward the distal end 414 of the needle 412 in a longitudinal direction. The actuated tip 435 then contacts and ejects the marker 450 into one or more of a slot 418 or an opening 422 of the needle 412 to mark a location of a biopsy in the target area.

Like the previously described actuators 144, 244, 344, the actuator 437 may include one or more of a plunger-type mechanism and/or a spring-loaded actuator button, the structure of which is described above relative to actuators 144, 244. In operation, in any case, the actuator 437 provides a force in a longitudinal direction toward the distal end 414 of the needle 412 that counters and is greater than the force of the spring (not shown) of the tip 435 that biases the tip 435 in an opposite direction, e.g., a longitudinal direction toward the proximal end 416 of the needle 212. Thus, upon actuation of the actuator 437, the tip 435 of the cutting apparatus 430 moves, e.g., springs, in a longitudinal direction toward the marker 450, thereby ejecting the marker out of one or both the slot 418 or the opening 422 to mark the location of the tissue biopsy. Upon removal of the force from the actuator 437 on the tip 435, the tip 435 springs back into an original position, which is spring-biased in a longitudinal direction toward the proximal end 416 of the needle 412.

While the slot 418 depicted in FIG. 9 is disposed on a top outside surface of the body 413 of the needle 412, one of ordinary skill in the art will appreciate that the slot 418 may alternatively be disposed on another outside surface of the needle 412, such as a lower outside surface, and still fall within the scope of the present disclosure. In addition, while the actuator 437 is disposed on an inside surface of the handle 424, one of ordinary skill in the art will appreciate that the actuator 437 may alternatively be disposed on any other surface or area of the handle and still fall within the scope of the present disclosure.

While not depicted in FIGS. 8A, 8B or 9, the cocking mechanism 260 may also be included on the device 300, 400. For example, the cocking mechanism 260 may be secured to the handle 324, 424 adjacent to the actuator 344, 437 such that upon actuation of the actuator 344, 437, in one example, a marker from the plurality of markers of the cocking mechanism 260 is pulled into the lower chamber 374 or sole chamber of the needle 312, 412 and pushed to the distal end 314, 414. In addition, the cocking mechanism 260 may be secured to the handle 324, 424 at one of a proximal end 328, 428 of the handle 324, 424 or between the proximal end 328, 428 of the handle 324, 424 and the proximal end 316, 416 of the needle 312, 412. The cocking mechanism 260 will operate with the device 300, 400 in the same manner described above relative to the operation of the cocking mechanism 260 with the device 200, for example.

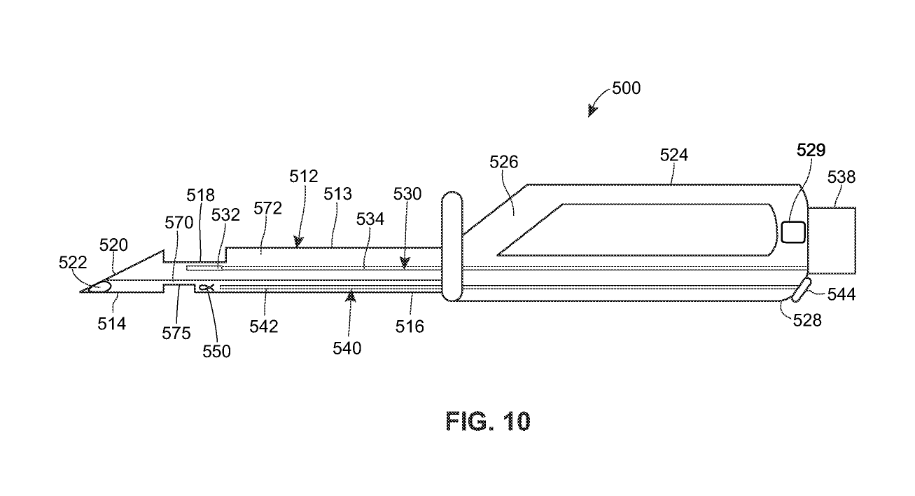

Referring now to FIG. 10, a biopsy device 500 according to another aspect of the present disclosure is depicted. The biopsy device 500 is similar to the biopsy device 300 of FIG. 8A. In fact, elements and structural components of the biopsy device 500 common or similar to elements and structural components of the biopsy device 300 include reference numerals 200 more than the biopsy device 300.

For example, like the biopsy device 300 of FIG. 8A, the biopsy device 500 is adapted to be inserted into a target area (not shown) of a tumor, for example. The biopsy device 500 also includes a needle 512 having a body 513 with a distal end 514 and a proximal end 516 disposed opposite the distal end 514. A slot 518 is disposed adjacent the distal end 514 of the needle 512, and the needle 512 further includes a tip 520 having an opening 522. While the slot 518 of the biopsy device 500 is disposed on an outer surface of the body 513 of the needle 512 and is generally rectangular in shape, one of ordinary skill in the art will appreciate that the slot 518 may alternatively be semi-circular, circular, triangular, or rounded in shape and still fall within the scope of the present disclosure.

As further depicted in FIG. 10, the biopsy device 500 also includes a handle 524 having a distal end 526 coupled to the proximal end 516 of the needle 512 and a proximal end 528 Like the handle 312 of the biopsy device 300, the handle 512 may also include a motor 529 adapted to drive a cutting mechanism and a vacuum (not shown) disposed within the biopsy device 500, such as the handle 524.

In addition, and like the needle 312, the needle 512 of the device 500 has a double barrel configuration. More specifically, and as depicted in FIG. 10, the needle 512 includes a septum 570 disposed within a bore of the body 513 of the needle 512. The septum 570 extends along the length of the needle 512 from the distal end 514 to the proximal end 516, creating an upper chamber 572 and a lower chamber 574 within the needle 512. The upper chamber 572 receives a cutting mechanism 530 and tissue excised from a target area during the biopsy. The lower chamber 574 is disposed below and opposite the upper chamber 572 and includes a preloaded marker 550 and an ejector rod 542 of an ejector mechanism 540. The ejector mechanism 540 includes an actuator 544, such as an actuator button, disposed on the handle 524 and operatively connected to the ejector rod 542. The actuator 544 may include one or more of a plunger mechanism or a spring-loaded actuator 544, such as the previously described actuator 344, and, therefore, may further operate like the actuator 344 described above.

In this example, the upper chamber 572 is larger than the lower chamber 574, such that the upper chamber 572 is able to receive a significant amount of excised tissue during a biopsy. In one example, the upper chamber 572 includes a 10-gauge aperture, and the lower chamber 574 includes a 14 or a 15-gauge aperture, or a similar size just large enough to accommodate the ejector rod 542. The upper and lower chambers 572, 574 may alternatively include various other sizes of apertures, in particular variations in which the upper chamber 572 is larger than the lower chamber 574, and still fall within the scope of the disclosure.

Unlike the needle 312 of the device 300, for example, the device 500 also includes an undersurface slot 575, e.g., a second slot 575. The undersurface slot 575 is disposed below the slot 518, e.g., the cutting slot, the biopsy slot, on an outer surface of the needle 512. The undersurface slot 575 opens into the lower chamber 574 of the double barrel configuration needle 512. While the slot 575 is generally rectangular in shape, one of ordinary skill in the art will appreciate that the slot 575 may alternatively be various other shapes, such as one or more of semi-circular, circular, or triangular in shape, for example, and still fall within the scope of the present disclosure.

Upon completion of a biopsy, e.g., when an adequate amount of tissue from the target area of the tumor has been extracted through the upper chamber 572 of the needle 512, the actuator 544 may be actuated to position a marker at the point of incision at the target area of the tumor. More specifically, a marker, such as the marker clip 550, is preloaded in the distal end 514 of the needle 512. Upon actuation of the actuator 544, the ejector rod 542 moves toward the distal end 514 of the needle 512 to contact the marker clip 550 and push and/or eject the marker clip 550 through the undersurface slot 575 and into the target area of the tumor. Thus, the undersurface slot 575 allows the marker clip 550 to be deployed out of a lower surface, such as a bottom, outer surface, or an undersurface, of the needle 512 instead of out through the hole 522, for example.

While the undersurface slot 575 is disposed on an outer surface of the double barrel configuration needle 512, one of ordinary skill in the art will understand and appreciate that the undersurface slot may alternatively or additionally be disposed on an outer surface, e.g., an undersurface, of a single barrel configuration needle. Such single barrel configuration needles include the needles 112, 212, 412, 612, and 712, depicted in FIGS. 4, 7, 9, 11A, and 12A, respectively. In addition, as one of ordinary skill in the art will further appreciate, the undersurface slot disposed on one or more of the single barrel configuration needles 112, 212, 412, 612, and 712 will operate in the same manner as the undersurface slot 575 disposed on the outside surface of the double barrel configuration needle 512 (FIG. 10). More specifically, and for example, upon actuation of an actuator of any of the biopsy devices 100, 200, 400, 600, 700, an ejector rod may push the marker through the undersurface slot and into the target area.

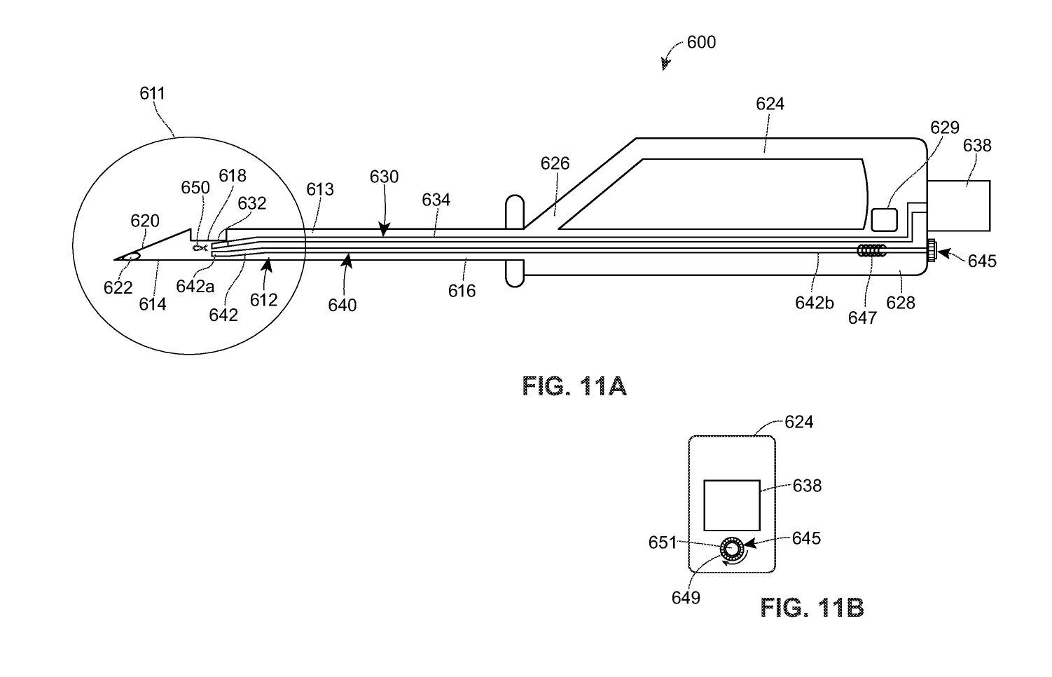

Referring now to FIG. 11A, a biopsy device 600 of the present disclosure adapted to be inserted into a target area 611 of a tumor, for example, is depicted. The biopsy device 600 is similar to the biopsy device 100 of FIG. 4. In fact, elements and structural components of the biopsy device 600 common or similar to elements and structural components of the biopsy device 100 include reference numerals 500 more than the biopsy device 100.

For example, like the biopsy device 100 of FIG. 4, the biopsy device 600 includes a needle 612 having a body 613 with a distal end 614 and a proximal end 616 disposed opposite the distal end 614. A slot 618, such as a cutter slot or a biopsy slot, is disposed adjacent the distal end 614 of the needle 612 and on an outer side of the needle 612, such as an outer top side. While the slot 618 is generally rectangular in shape, one of ordinary skill in the art will appreciate that the slot 618 may alternatively be various other shapes, such as one or more of semi-circular, circular, cylindrical, or triangular in shape, for example, and still fall within the scope of the present disclosure. The needle 612 further includes a tip 620 also disposed adjacent the distal end 614 of the needle 612. The tip 620 includes an opening 622 adapted to receive a marker, such as a marker clip 650, in one example, as described in more detail below.

The biopsy device 600 further includes a handle 624 having a distal end 626 that is coupled to the proximal end 616 of the needle 612, and a proximal end 628. A biopsy motor 629 may be disposed within the handle 624 at the proximal end 628 to provide power to a vacuum (not shown), for example, and/or a cutting apparatus of the biopsy device 600, as described more below. The vacuum may provide a suction force at the proximal end of the needle 612 to withdraw tissue from the point of incision in the target area 611 into the needle 612. While the handle 624 is depicted as generally rectangular in shape in FIG. 11A, the handle 624 may take the shape of one or more of a variety of shapes, such as a semi-circle, a circle, an oval, a cylinder, or a triangle, and still fall within the scope of the present disclosure.

As further depicted in FIG. 11A, the biopsy device 600 also includes a cutting apparatus 630 disposed within the body 613 of the needle 612. The cutting apparatus 630 includes a cutting mechanism 632 disposed adjacent the distal end 614 of the needle 612 and a cutter drive 634 coupled to the cutting mechanism 632. In one example, the cutting mechanism 632 is disposed adjacent to the slot 618, such that the cutting mechanism 632 rotates and advances in the slot 618 during operation of the biopsy device 600 to remove tissue from the target area 611 through the slot 618, for example. The cutter drive 634 extends along the length of the needle 612 of the biopsy device 600 from the cutting mechanism 632 disposed at the distal end 614 of the needle 612 to the proximal end 616 of the needle 612. In addition, a tissue recovery chamber 638 is disposed at the proximal end 628 of the handle 624 for receiving excised tissue from the needle 612.

The biopsy device 600 further includes an ejector mechanism 640 having an ejector rod 642 disposed within the body 613 of the needle 612 and an actuator 645, such as a button, disposed on the handle 624 and operatively coupled to the ejector rod 642. In one example, and as depicted in FIG. 11A, the actuator button 645 is disposed on a rear end of the handle 624 near the proximal end 628 of the handle 624 for ease of actuation during use of the biopsy device 600. Alternatively, the actuator button 645 may be disposed on another side or area of the handle 624 and still fall within the scope of the present disclosure.

In one example, the actuator button 645 includes a winding mechanism 649 having a depressor 651 disposed in a center of the winding mechanism 649, as depicted in FIG. 11B. The winding mechanism 649 is circular in shape, adapted to be rotated or wound in a clockwise direction, and spring-loaded. More specifically, and in one example, a spring 647 is disposed on the ejector rod 642 near the proximal end 628 of the handle 624 and is operatively coupled to the actuator button 645. When the winding mechanism 649 is rotated or wound in a clockwise direction, e.g., such as one or more of a half-turn, one full turn, or two full turns, the spring 647 moves to a compressed position and the actuator button 645 moves to a loaded position, such as a spring-loaded position.

When it is time to deploy the pre-loaded marker 650 disposed in the needle 612 through one or more of the slot 618 or the opening 622, the depressor 651 of the winding mechanism 649 is pushed in a direction toward the distal end 614 of the needle 612. This force counteracts and overcomes the force of the loaded spring 647 disposed on the ejector rod 642, causing the spring 647 to extend in a direction toward the distal end 614 of the needle 612 and move the ejector rod 642 in the same direction. A distal end 642a of the ejector rod 642 then contacts and pushes the marker 650 to move the marker 650 one of through the slot 618 or the opening 622. Said another way, upon pushing, e.g., actuating, the depressor 651, the ejector rod 642 is thrown forward toward the distal end 614 of the needle 612 to eject the marker 650 out of the slot 618 or the opening 622.

Like the needle 112 of the biopsy device 100, the needle 612 of the biopsy device 600 of FIG. 11A may include one of a 12-gauge, a 13 gauge, or a 14-gauge aperture disposed along the length of the body 613, such that an adequate amount of tissue extracted from the target area 611 by the cutting mechanism 632 may be suctioned through the needle 612. In addition, the aperture of the body 613 of the needle 612 must further include a diameter large enough to accommodate both the ejector rod 642 of the ejector mechanism 640 and the cutting mechanism 630, as depicted in FIG. 11A, for example.

While the winding mechanism 649 is disposed on the biopsy device 600, one of ordinary skill in the art will understand and appreciate that the winding mechanism 649 may alternatively or additionally be disposed on any of the other biopsy devices 100, 200, 300, 400, 500 and 700 depicted, for example, in FIGS. 4, 7, 8A, 9, 10, and 12A and still fall within the scope of the pending claims. In addition, one of ordinary skill in the art will further appreciate that the winding mechanism 649 disposed on any one or more of the biopsy devices 100, 200, 300, 400, 500, and 700 will operate in the same manner described above relative to the biopsy device 600.

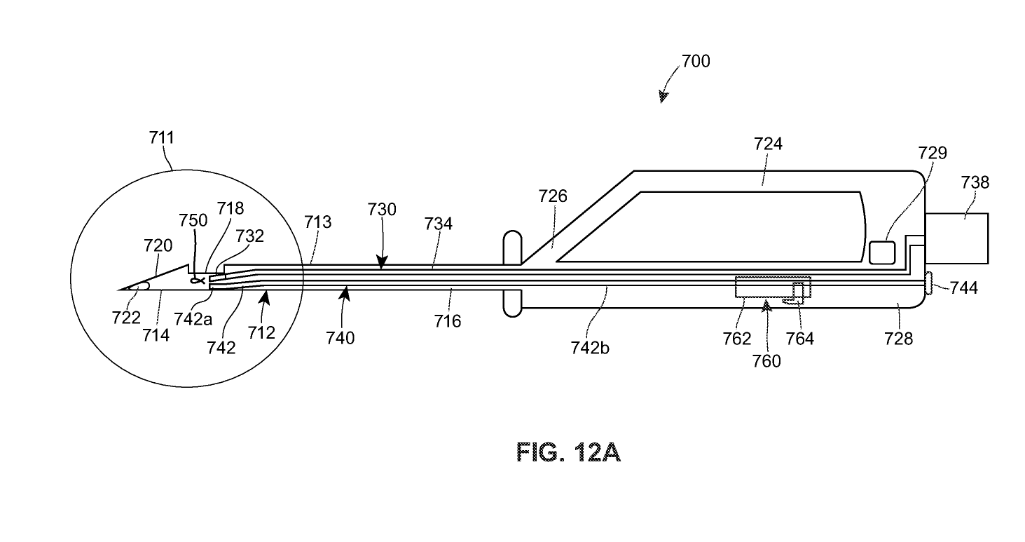

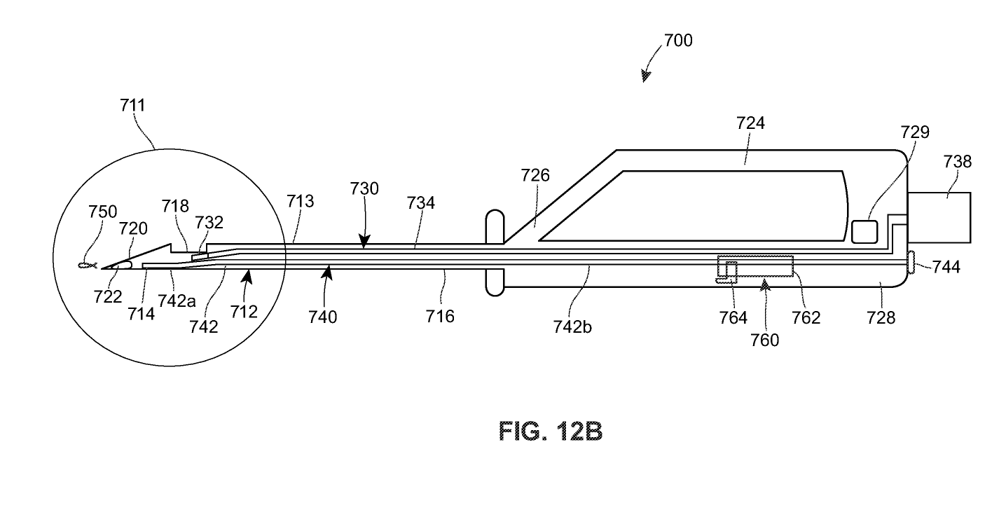

Referring now to FIGS. 12A and 12B, a biopsy device 700 according to another aspect of the present disclosure is depicted. The biopsy device 700 is also adapted to be inserted into a target area 711 of a tumor, for example, and is similar to the biopsy device 100 of FIG. 4. In fact, elements and structural components of the biopsy device 700 common or similar to elements and structural components of the biopsy device 100 include reference numerals 600 more than the biopsy device 100.

For example, like the biopsy device 100 of FIG. 4, the biopsy device 700 includes a needle 712 having a body 713 with a distal end 714 and a proximal end 716 disposed opposite the distal end 714. A slot 718, such as a cutter slot, is disposed adjacent the distal end 714 of the needle 712 and on an outer side of the needle 712, such as an outer, top side of the needle 712. While the slot 718 is generally rectangular in shape, one of ordinary skill in the art will appreciate that the slot 718 may alternatively be various other shapes, such as one or more of semi-circular, circular, or triangular in shape, for example, and still fall within the scope of the present disclosure. The needle 712 further includes a tip 720 also disposed adjacent the distal end 714 of the needle 712. The tip 720 includes an opening 722 adapted to receive a marker, such as a marker clip 750, in one example, as described in more detail below.

The biopsy device 700 further includes a handle 724 having a distal end 726 that is coupled to the proximal end 716 of the needle 712, and a proximal end 728. A biopsy motor 729 may be disposed within the handle 724 at the proximal end 728 to provide power to a vacuum (not shown), for example, and/or a cutting apparatus of the biopsy device 700, as described more below. The vacuum may provide a suction force at the proximal end of the needle 712 to withdraw tissue from the point of incision in the target area 711 into the needle 712. While the handle 724 is depicted as generally rectangular in shape in FIGS. 12A and 12B, the handle 724 may take the shape of one or more of a variety of shapes, such as a semi-circle, a circle, a cylinder, an oval, or a triangle, and still fall within the scope of the present disclosure.

As further depicted in FIG. 12A, the biopsy device 700 also includes a cutting apparatus 730 disposed within the body 713 of the needle 712. The cutting apparatus 730 includes a cutting mechanism 732 disposed adjacent the distal end 714 of the needle 712 and a cutter drive 734 coupled to the cutting mechanism 732. In one example, the cutting mechanism 732 is disposed adjacent to the slot 718, such that the cutting mechanism 732 rotates and advances in the slot 718 during operation of the biopsy device 700 to remove tissue from the target area 711 through the slot 718, for example. The cutter drive 734 extends along the length of the needle 712 of the biopsy device 700 from the cutting mechanism 732 disposed at the distal end 714 of the needle 712 to the proximal end 716 of the needle 712. In addition, a tissue recovery chamber 738 is disposed at the proximal end 728 of the handle 724 for receiving excised tissue from the needle 712.

The biopsy device 700 further includes an ejector mechanism 740 having an ejector rod 742 disposed within the body 713 of the needle 712 and an actuator 744, such as a button, disposed on the handle 724 and operatively coupled to the ejector rod 742. In one example, and as depicted in FIG. 12A, the actuator button 744 is disposed on a rear end of the handle 724 near the proximal end 728 of the handle 724 for ease of actuation during use of the biopsy device 700. Alternatively, the actuator button 744 may be disposed on another side or area of the handle 724 and still fall within the scope of the present disclosure.

In one example, the actuator button 744 is operatively coupled to a racking cocking mechanism 760. The racking cocking mechanism 760 includes a side slot 762 disposed on a side portion of the handle 724. A finger trigger 764 is disposed within and extends through the side slot 762 and is operatively coupled to the ejector rod 742. When the finger trigger 764 is moved in a direction toward the actuator button 744 or the proximal end 728 of the handle 724, this movement of the finger trigger 764 in turn moves the ejector rod 742 in the same direction and a same distance the finger trigger 764 is moved, thereby cocking the ejector rod 742, as depicted in FIG. 12A. Said another way, the ejector rod 742 and the finger trigger 764 are in a cocked, e.g., such as a compressed position or unextended position, in FIG. 12A. More specifically, upon pulling back the finger trigger 764 in a direction toward the proximal end 728 of the handle 724, a spring (not shown) or other similar mechanism is loaded to the ejector rod 742, resulting in the cocked, e.g., unextended, position of the finger trigger 764 and ejector rod 742.

Referring now to FIG. 12B, when it is time to deploy the pre-loaded marker 750 disposed in the needle 712 through one or more of the slot 718 or the opening 722, the actuator button 744 is pushed in a direction toward the distal end 714 of the needle 712. Upon depressing the actuator button 744, the finger trigger 764 and the ejector rod 742 operatively coupled thereto both move forward, e.g., fire, toward the distal end 714 of the needle 712. A distal end of the ejector rod 742 then contacts the marker 750 upon actuation, e.g., being pushed, to eject the marker clip 750 through the slot 718 or the opening 722 and mark the location of the biopsy. Said another way, the ejector rod 742 and the finger trigger 764 are both in an uncocked or extended position in FIG. 12B, with the finger trigger 764 moved toward an end of the side slot 762 nearest to the distal end 714 of the needle 712.

To refire and/or deploy another marker to mark another location of the biopsy, the needle 712 may be removed or detached from the handle 724 and a new needle having another pre-loaded marker may be attached to the handle 724. The finger trigger 764 may then again be pulled back in a direction toward the proximal end of the handle 724 to cock or load the ejector rod 742 and the finger trigger 764. When it is again time to deploy the pre-loaded marker disposed in the second needle through one or more of the slot 718 or the opening 722, the actuator button 744 is again pushed in a direction toward the distal end 714 of the needle 712, allowing the marker clip to be ejected out of the needle to mark the location.

While the racking cocking mechanism 760 is disposed on the biopsy device 700 depicted in FIG. 12A, for example, one of ordinary skill in the art will understand and appreciate that the racking cocking mechanism may alternatively or additionally be disposed on any of the other biopsy devices 100, 200, 300, 400, 500, and 600 depicted, for example, in FIGS. 4, 7, 8A, 9, 10, and 11A, and still fall within the scope of the pending claims. In addition, one of ordinary skill in the art will further appreciate that the racking cocking mechanism 760 disposed on any one or more of the biopsy devices 100, 200, 300, 400, 500 and 600 will operate in the same manner described above relative to the biopsy device 700.

In each of the biopsy devices 100, 200, 300, 400, 500, 600, 700, while the actuators 144, 244, 344, 437, 544, 645, 744 are referred to as buttons in one example, the actuators 144, 244, 344, 544, 645, 744 may alternatively be other actuation mechanisms, such as any of the actuation mechanisms of any other examples in the application, and not depart from the scope of the present disclosure. For example, the actuator 144, 244, 344, 437, 544, 645, 744 may be a plunger, a post, a pusher, a plate, a flange, or any other structural component capable of actuating the ejector rod 142, 242 or the tip 435 of the cutting apparatus 430. Still further, the actuator 144, 244, 344, 437, 544, 645, 744 may alternatively be an electronic actuator, such as an electronic surface and/or a sensor and still fall within the scope of the present disclosure.

In addition, while the markers depicted are marker clips 150, 250, 350, 450, 550, 650, 750, various other markers may alternatively be used and still fall within the scope of the present disclosure. For example, the marker may be also be a marker tag, a marker label, a marker post or any other marker capable of adhering to tissues or cells in a target area to mark a point or points of incision. Moreover, the marker may include a material or a substrate that is biodegradable and/or dissolvable, such that after a period of time, the marker safely dissolves within the target area of the tumor and removal of the marker is not required.

In operation, the biopsy devices 100, 200, 300, 400, 500, 600, 700 may be utilized to both biopsy a target area of a tumor and deploy a marker into the same target area in one step without having to remove the biopsy device 100, 200, 300, 400, 500, 600, 700 from an insertion site to later deploy any marker. More specifically, the biopsy devices 100, 200, 300, 400, 500, 600, 700 may employ the following biopsy method in one example operation. The biopsy method includes first inserting the biopsy device 100, 200, 300, 400, 500, 600, 700 into tissue of a target area of a tumor. The method further comprises actuating a cutter mechanism 132, 232, 332, 442, 542, 642, 742 of a cutting apparatus 130, 230, 330, 430, 530, 630, 730 disposed within the biopsy device 100, 200, 300, 400, 500, 600, 700 to remove tissues or cells of the target area 111, 211, 311, 611, 711 and actuating an actuator 144, 244, 344, 544, 645, 744 of one of an ejector mechanism 140, 240 or a cutting mechanism 430. Upon actuation of the actuator 144, 244, 344, 437, 544, 645, 744, the method may further include ejecting a marker, such as a marker clip 150, 250, 350, 450, 550, 650, 750 disposed adjacent the distal end 114, 214, 314, 414, 514, 614, 714 of the needle 112, 212, 312, 412, 512, 612, 712 through one or more of an opening 122, 222, 322, 422, 522, 622, 722 or a slot 118, 218, 318, 418, 518, 618, 718 disposed adjacent the distal end 114, 214, 314, 414, 514, 614, 714 of the needle 112, 212, 312, 412, 512, 612, 712 to mark a point of incision in tissue of the target area, e.g., mark a location of a biopsy in the target area.

The biopsy method may further comprise preloading at least one marker 150, 250, 350, 450, 550, 650, 750 in the distal end 114, 214, 314, 414, 514, 614, 714 of the needle 112, 212, 312, 412, 512, 612, 712 before inserting the biopsy device 100, 200, 300, 400, 500, 600, 700 into the tissue of the target area. In one example, preloading the marker clip 150, 250, 350, 450 comprises inserting a marker deployment device having a catheter with the marker clip 150, 250, 350, 450, 550, 650, 750 disposed therein into the needle 112, 212, 312, 412, 512, 612, 712 until the at least one marker clip 150, 250, 350, 450, 550, 650, 750 is positioned in the distal end 114, 214, 314, 414, 514, 614, 714 of the needle 112, 212, 312, 412, 512, 612, 712. In another example, actuating the actuator button 144, 244 may include removing at least one marker clip 150, 250 from a plurality of marker clips 264 preloaded into a chamber 262 of a cocking mechanism 260 coupled to the handle 124, 224. The at least one marker clip 150, 250 is then pushed to the distal end 114, 214 of the needle 112, 212 via an ejector rod 142, 242 of an ejector mechanism 140, 240.

In view of the foregoing, one of ordinary skill in the art will appreciate that the biopsy devices 100, 200, 300, 400, 500, 600, 700 of the present disclosure offer several advantages over known biopsy devices and methods that require separate marking devices to mark a target area of a tumor. For example, by including a preloaded marker within the needle 112, 212, 312, 412 and, in some examples, an ejector mechanism 140, 240, 340 or a tip 435 of a cutting apparatus 430 that pushes the marker out of the slot 118, 218, 318, 418, 518, 618, 718 or the opening 122, 222, 322, 422, 522, 622, 722, the biopsy procedure and the marking procedure become a one step process. In other words, a separate marking deployment device is no longer needed to effect marking in the target area 111, 211, 611, 711. Therefore, it is not necessary for an operator of the biopsy devices 100, 200, 300, 400, 500, 600, 700 to first remove the biopsy device 100, 200, 300, 400, 500, 600, 700 from the incision site and then insert a marker deployment device back into the target area of the tumor, for example. As a result, the marker accuracy is significantly improved because it may be placed virtually automatically and nearly simultaneously after the needed tissues are excised from the target area. Various opportunities for human error in placement of the conventional separate deployment device in the target area to mark the point of incision are eliminated, as there is no need to remove the biopsy device 100, 200, 300, 400, 600, 700 from the target area 111, 211, 611, 711 for example. Moreover, because the marking process is condensed with the biopsy process, the biopsy time is reduced and the procedure becomes more efficient.

Still further, the new cocking mechanism provides a more efficient method of preloading the needle 112, 212, 312, 412, 512, 612, 712 with the marker. In other words, the cocking mechanism 260 may be actuated to remove a clip 250 from the cocking mechanism 260 and push the clip 250 to the distal end 214 of the needle 212 without having to remove the biopsy device 200.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Some implementations may be described using the expression “coupled” along with its derivatives. For example, some implementations may be described using the term “coupled” to indicate that two or more elements are in direct physical or electrical contact. The term “coupled,” however, may also mean that two or more elements are not in direct contact with each other, but yet still co-operate or interact with each other. The implementations are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the implementations herein. This is done merely for convenience and to give a general sense of the invention. This description should be read to include one or at least one and the singular also includes the plural unless it is obvious that it is meant otherwise.

Further, while particular implementations and applications have been illustrated and described, it is to be understood that the disclosed implementations are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims. the present application.

1. A device for marking a location of a tissue biopsy, the device comprising:
a needle including a proximal end, a distal end disposed opposite the proximal end, a slot disposed adjacent the distal end, and a tip disposed adjacent the distal end and having an opening;
a handle coupled to the proximal end of the needle;
a cutting apparatus including a cutting mechanism disposed within the needle, the cutting mechanism disposed adjacent the distal end of the needle and the slot, the cutting apparatus adapted to remove tissue from the target area through the slot; and
an ejector mechanism, the ejector mechanism including an ejector rod disposed within the needle and an actuator disposed on the handle and operatively coupled to the ejector rod;
wherein, upon removal of the tissue from the target area, the actuator of the ejector mechanism is actuated to eject a marker disposed within the distal end of the needle through one or more of the slot or the opening to mark a location of a tissue biopsy.

2. The device of claim 1, wherein the actuator is disposed on one of a thumb side of the handle or on or near a proximal end of the handle.

3. The device of claim 1, further comprising a cocking mechanism removably attached to the handle and having a chamber preloaded with a plurality of markers, the cocking mechanism coupled to the actuator, such that when the actuator is actuated, one marker from the plurality of markers disposed within the cocking mechanism is adapted to be removed from the cocking mechanism, pushed into the needle and ejected out of one of the slot or the opening disposed at the distal end of the needle.

4. The device of claim 3, wherein the cocking mechanism is coupled to the handle at one of a proximal end of the handle or between the proximal end of the needle and the proximal end of the handle.

5. The device of claim 1, wherein the marker is preloaded in the needle adjacent the distal end and adapted to mark a point of incision in a target area.

6. The device of claim 1, wherein the marker is one or more of a marker clip, a marker label, or a marker tag, the marker including dissolvable material not requiring removal from the target area.

7. The device of claim 1, wherein the cutting apparatus further includes a cutter drive disposed within a body of the needle, the cutter drive disposed adjacent to the ejector rod.

8. The device of claim 1, wherein the handle further comprises a motor disposed near the proximal end of the handle, the motor operatively coupled to the cutting apparatus to actuate the cutting mechanism during the biopsy.

9. A device for marking a location of a tissue biopsy, the device comprising:
a needle including a body with an upper chamber and a lower chamber, the needle further including a proximal end, a distal end disposed opposite the proximal end, a slot disposed adjacent the distal end, and a tip disposed adjacent the distal end and having an opening;
a handle coupled to the proximal end of the needle;
a cutting apparatus including a cutter mechanism disposed within the upper chamber of the needle, the cutter mechanism adjacent the distal end of the needle, the cutting apparatus adapted to remove tissue from the target area through the slot and the upper chamber adapted to receive the removed tissue; and
an ejector mechanism including an ejector rod disposed within the lower chamber of the needle and an actuator disposed on the handle and operatively coupled to the ejector rod;
wherein, upon removal of the tissue from the target area, the actuator of the ejector mechanism is actuated to eject a marker disposed within the lower chamber through the needle to mark a location of a tissue biopsy.

10. The device of claim 9, the device further comprising a cocking mechanism removably attached to the handle and having a chamber preloaded with a plurality of markers adapted to mark a point of incision in a target area, the cocking mechanism coupled to the actuator, such that when the actuator is actuated, one marker from the plurality of markers disposed within the cocking mechanism is removed from the cocking mechanism and ejected out of the opening disposed at the distal end of the needle.

11. The device of claim 10, wherein when the actuator is actuated and after the at least one marker from the plurality of markers disposed within the cocking mechanism is removed from the cocking mechanism, the at least one marker is pushed into a body of the needle via the ejector rod to the lower chamber of the needle adjacent the distal end.

12. The device of claim 10, wherein the cocking mechanism is coupled to the handle one or more of at a proximal end of the handle or between the proximal end of the needle and the proximal end of the handle.

13. The device of claim 9, wherein the cutting apparatus further includes a cutter drive coupled to the cutter mechanism and disposed within the upper chamber of the needle.

14. The device of claim 9, further comprising an undersurface slot on an outer surface of the needle, the undersurface slot opening into the lower chamber, wherein when the actuator is actuated, the marker disposed within the lower chamber is ejected through the undersurface slot to mark the location of the tissue biopsy.

15. A method for marking a location of a tissue biopsy, the method comprising:
inserting a biopsy device into a target area, the biopsy device including a needle and a handle operatively coupled to a proximal end of the needle;
actuating a cutting mechanism of a cutting apparatus, the cutting mechanism disposed within the needle to remove tissue from the target area;
actuating an actuator of one of an ejector mechanism or the cutting apparatus, the actuator disposed on the handle, and the ejector mechanism including an ejector rod disposed within the needle of the biopsy device, the ejector rod operatively coupled to the actuator of the ejector mechanism; and
ejecting a marker disposed adjacent the distal end of the needle through one or more of an opening of a tip of the needle, a slot disposed adjacent the distal end of the needle, or a slot disposed on a lower surface of the needle to mark a location of a tissue biopsy.

16. The method of claim 15, further comprising preloading at least one marker adjacent the distal end of the needle before inserting the biopsy device into the target area.

17. The method of claim 16, wherein preloading the at least one marker comprises inserting a catheter with the at least one marker into the needle until the at least one marker is positioned adjacent the distal end of the needle.

18. The method of claim 15, wherein actuating the actuator of one of the ejector mechanism or the cutting apparatus comprises removing at least one marker from a plurality of markers preloaded in a chamber of a cocking mechanism coupled to the handle, and pushing the at least one marker adjacent the distal end of the needle.

19. The method of claim 15, wherein actuating the actuator of the ejector mechanism comprises pushing the actuator, thereby moving the ejector rod in a longitudinal direction toward the distal end of the needle to contact the marker.

20. The method of claim 15, wherein ejecting a marker disposed adjacent the distal end of the needle comprises ejecting one of a marker clip, a marker tag, a marker post, a marker label and/or any other marker adapted to mark a location of a tissue biopsy.

21. A device for marking a location of a tissue biopsy, the device comprising:
a needle including a proximal end, a distal end disposed opposite the proximal end, a slot disposed adjacent the distal end, and a tip disposed adjacent the distal end and having an opening;
a handle coupled to the proximal end of the needle; and
a cutting apparatus including a cutting mechanism disposed within the needle, a tip disposed adjacent to and connected to the cutting mechanism, and an actuator operatively coupled to the cutting mechanism and disposed on the handle, the cutting mechanism disposed adjacent the distal end of the needle and the slot, the cutting apparatus adapted to remove tissue from the target area through the slot;
wherein, upon removal of the tissue from the target area, the tip of the cutting apparatus is actuated by the actuator to eject a marker disposed within the distal end of the needle through one or more of the slot or the opening to mark a location of a tissue biopsy.

22. The device of claim 21, where the tip of the cutting apparatus is spring-loaded.

23. The device of claim 1, wherein the actuator is a winding mechanism having a depressor disposed in a center of the winding mechanism, and a spring is disposed on the ejector rod near the proximal end of the handle, the spring operatively coupled to the actuator such that when the winding mechanism is rotated in a clockwise direction the actuator is in a spring-loaded position, and when the depressor is pushed the spring and the ejector rod move in a direction toward the distal end of the needle to eject the marker through one of the slot or the opening.

24. The device of claim 1, further comprising a racking cocking mechanism, the racking cocking mechanism having a side slot disposed on a side portion of the handle, and a finger trigger disposed within the side slot and operatively coupled to the ejector rod, wherein movement of the finger trigger in a direction toward the proximal end of the handle puts the ejector rod and finger trigger in a cocked position, and pushing the actuator button in a direction toward the distal end of the needle moves the ejector rod and finger trigger to an uncocked position to eject the marker through one or more of the slot or the opening.

25. The method of claim 15, wherein actuating the actuator of one of the ejector mechanism or the cutting apparatus comprises pushing an actuator button in a direction toward the distal end of the needle to move the ejector rod and a finger trigger disposed within a side slot of a racking cocking mechanism to an extended position.

26. The method of claim 15, further comprising actuating the actuator of the ejector mechanism, wherein actuating the actuator of the ejector mechanism comprises rotating a winding mechanism in a clockwise direction to cock the ejector rod and the actuator, and pushing a depressor disposed in a center of the winding mechanism to uncock the ejector rod and the actuator.

27. The device of claim 9, wherein the actuator is a winding mechanism having a depressor disposed in a center of the winding mechanism, and a spring is disposed on the ejector rod near the proximal end of the handle, the spring operatively coupled to the actuator such that when the winding mechanism is rotated in a clockwise direction the actuator is in a spring-loaded position, and when the depressor is pushed the spring and the ejector rod move in a direction toward the distal end of the needle to eject the marker through one of the slot or the opening.

28. The device of claim 9, further comprising a racking cocking mechanism, the racking cocking mechanism having a side slot disposed on a side portion of the handle, and a finger trigger disposed within the side slot and operatively coupled to the ejector rod, wherein movement of the finger trigger in a direction toward the proximal end of the handle puts the ejector rod and finger trigger in a cocked position, and pushing the actuator button in a direction toward the distal end of the needle moves the ejector rod and finger trigger to an uncocked position to eject the marker through one or more of the slot or the opening.

29. The device of claim 1, further comprising an undersurface slot on an outer surface of the needle, the undersurface slot opening into the needle, wherein when the actuator is actuated, the marker is ejected through the undersurface slot to mark the location of the tissue biopsy.
